(19) United States
(12) Patent Application Publication
(10) Pub. No.: US 2019/0111222 A1
(43) Pub. Date: Apr. 18, 2019

(54) DEVICE FOR MEASURING BREATHING CAPACITY/COUNTING INHALATION

(71) Applicant: VIRGILANT TECHNOLOGIES LIMITED, Hong Kong (CN)

(72) Inventors: Chung Sheng WANG, Hong Kong (CN); Jonathan David Piteo Tarpy, Hong Kong (CN)

(21) Appl. No.: 16/161,922

(22) Filed: Oct. 16, 2018

(30) Foreign Application Priority Data
Oct. 17, 2017 (TW) 106135561

(51) Int. Cl. A61M 15/00

(52) U.S. Cl. CPC A61M 15/008

(57) ABSTRACT

Disclosed is a device for counting the number of inhalation while medicine bottle is inserted into an inhalator, or measuring breathing capacity while medicine bottle is not inserted. The device includes a shell body, a roller, magnetic pieces, a sensor, a blue tooth integrated circuit (IC), a battery unit, and a circuit board. The roller is provided in the shell body, the magnetic pieces are attached onto the roller, and the circuit board is fixed on the shell body. The battery unit supplies electric power. When medicine agent is sprayed from the medicine bottle or the shell body is blown, the roller to rotate, the magnetic pieces synchronously rotate, and the sensor detects variation of a magnetic flux. The blue tooth IC calculates the number of inhalation when the medicine bottle is connected, or calculates breathing capacity of the user when the medicine bottle is not connected.

### BACKGROUND OF THE INVENTION

### 1. Field of the Invention

The present invention relates to a device for measuring breathing capacity/counting inhalation, and more specifically to a device performing a breathing capacity measuring mode or an inhalation counting mode depending on whether the inhalator is connected to the medicine bottle, and further employing a plurality of magnetic pieces on a roller in collocation with a sensor to detect and calculate a breathing capacity or a number of inhalation.

### 2. The Prior Arts

For those who suffer from respiratory uncomfortableness, it is necessary to carry an inhalator to inhale specific agent for temporary mitigation, or for patients with dyspnea or respiratory disease, the agent is directly sprayed into the mouth for instant therapeutic effect.

The traditional inhalator generally has a function of recording the number of inhalation, and a counter provided on the inhalator is intended to count how many times the medicine bottle is pressed by the user. However, the counter still performs counting even when the medicine bottle is empty and pressed in vain. As a result, the counted number of inhalation is not exact.

Therefore, it is greatly to provide a new device for measuring breathing capacity/counting inhalation, which performs a breathing capacity measuring mode or an inhalation counting mode depending on whether the inhalator is connected to the medicine bottle, and further employs a plurality of magnetic pieces on a roller in collocation with a sensor to detect and calculate a breathing capacity or a number of inhalation, thereby overcoming the problems in the prior arts.

### SUMMARY OF THE INVENTION

The primary objective of the present invention is to provide a device for measuring breathing capacity/counting inhalation detachably connected to an inhalator and generally comprises a shell body, a roller, a plurality of magnetic pieces, a circuit board, a battery unit, a sensor, and a blue tooth integrated circuit (IC). The inhalator is connected to or not connected to a medicine bottle. Further, the medicine bottle contains a medical agent or a gas at a preset pressure, and a switch is provided for the medicine bottle to restore or release the medical agent or the gas.

Specifically, the shell body forms a cavity, and is provided with a inhalation end for a user to inhale or exhale (blow), a connection end for connecting an end of the inhalator, and the other end of the inhalator connecting or not connecting the medicine bottle for the user inhaling or blowing the shell body. Thus, the user can inhale the medical agent or blow through the inhalation end.

Further, the roller is provided on an inner wall of the cavity and dragged by an air flow through the cavity to perform a high speed rotation or a low speed rotation for a period of time. The magnetic pieces are attached onto and rotate synchronously with the roller such that the sensor detects variation of the magnetic flux in the space generated by the magnetic pieces.

The circuit board is fixed to the shell body, and the battery unit, the sensor, and the blue tooth IC are provided on the circuit board. The battery unit is connected to the sensor and the blue tooth IC, and supplies electric power for operation. The sensor is near the roller like at the location over the roller. The roller is not in contact with the magnetic pieces. The sensor can be a Hall sensor. When the magnetic pieces rotate, the sensor detects variation of the magnetic flux caused by the magnetic pieces, thereby generating and transferring the electrical signal.

The blue tooth IC receives and employs the sensed electrical signal to perform an inhalation counting process or a breathing capacity detection process, thereby generating and transferring an inhalation counting result or a breathing capacity data. Additionally, the inhalation counting result or the breathing capacity data is wireless transferred to the external receiving device by the blue tooth IC for performing a data process to store and display the inhalation counting result or the breathing capacity data.

More specifically, when the inhalator is connected to the medicine bottle and pressed, the switch of the medicine bottle is opened by the inhalator, and the medical agent and gas in the medicine bottle spontaneously sprays into the inhalator from the medicine bottle due to the substantial high pressure. Thus, the medical or gas is transferred into the mouth of the user from the inhalation end, and the user can directly inhale the medical agent or gas from the inhalation end. At this time, the roller and magnetic pieces operate at the low speed rotation, and the blue tooth IC employs the electrical signal from the sensor to counts and obtain the inhalation counting result indicating the number of inhalation by the user. In other words, the above operation is an inhalation counting mode.

Furthermore, when the inhalator is not connected to the medical bottle, if the user exhales or blows the inhalation end, the roller and the magnetic pieces performs the high speed rotation, and the blue tooth IC employs the electrical signal to detect and calculate the breathing capacity, thereby obtaining the breathing capacity data, representing the breathing capacity of the user. In other words, the above operation is a breathing capacity measuring mode.

Since the present invention depends on whether the inhalator is connected to the medicine bottle to performs the breathing capacity measuring mode at the high speed rotation or the inhalation counting mode at the low speed rotation, the overall structure is simple, and quite easy to implement and maintenance. In particular, the present invention is more reliable and endurable so as to possess excellent market competitiveness and industrial utility, thereby possibly replacing all traditional inhalators.

### BRIEF DESCRIPTION OF THE DRAWINGS

The present invention will be apparent to those skilled in the art by reading the following detailed description of a preferred embodiment thereof, with reference to the attached drawings, in which:

FIG. 1 is a view showing the device for measuring breathing capacity/counting inhalation according to the embodiment of the present invention;

FIG. 2 is a view showing the device performing the inhalation counting mode according to the embodiment of the present invention; and

FIG. 3 is a view showing the device performing the breathing capacity measuring mode according to the embodiment of the present invention.

### DETAILED DESCRIPTION OF THE PREFERRED EMBODIMENT

The accompanying drawings are included to provide a further understanding of the invention, and are incorporated in and constitute a part of this specification. The drawings illustrate embodiments of the invention and, together with the description, serve to explain the principles of the invention.

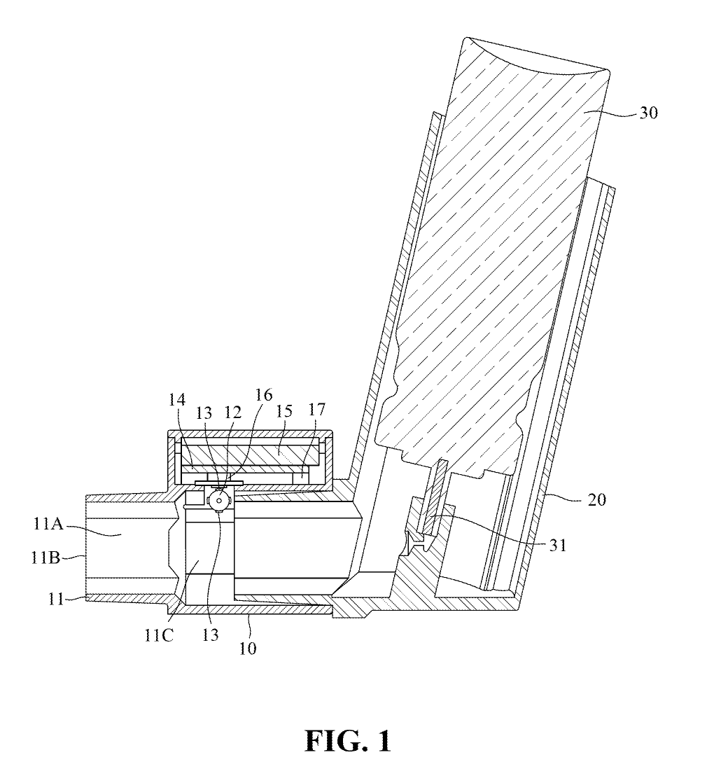

Please refer to FIG. 1 illustrating the device for measuring breathing capacity/counting inhalation according to the embodiment of the present invention. As shown in FIG. 1, the device 10 for measuring breathing capacity/counting inhalation according to the present invention generally comprises a shell body 11, a roller 12, a plurality of magnetic pieces 13, a circuit board 14, a battery unit 15, a sensor 16, and a blue tooth integrated circuit (IC) 17. Specifically, the device 10 is detachably connected to the inhalator 20, and the inhalator 20 is connected to or not connected to a medicine bottle 30.

For example, the inhalator 20 is substantially a hollow shell piece with an L-like shape, and comprises a horizontal part and an oblique part. The opening of the horizontal part is configured for connecting the shell body 11 of the device 10, and the opening of the oblique part is for connecting the medicine bottle 30. Therefore, when the device 10, the inhalator 20, and the medicine bottle 30 are connected together, a standalone body is formed and has an L-like shape.

In addition, the above medicine bottle 30 may contain specific medical agent of gas with a preset pressure higher than the normal atmosphere pressure like spray agent for curing respiratory disease or mitigating dyspnea, or highly concentrated oxygen gas. Further, the medicine bottle 30 comprises a switch 31 for controlling the medical agent or gas to be tightly sealed in the shell body 30 or released to the outside.

Specifically, the shell body 11 forms a cavity 11A, and has an inhalation end 11B and a connection end 11C. The inhalation end 11B is provided for the user to inhale or blow, the connection end 11C is configured for connecting one end of the inhalator 20, and the other end of the inhalator 2 is connected to or not connected to the medicine bottle 30. Additionally, the user can employ the inhalation end 11B to spray the medical agent or blow through the shell body 11.

The roller 12 is provided in the inner wall of the cavity 11A, and is dragged to perform a low speed rotation or a high speed rotation for a period of time by the air flow in the cavity 11A. The magnetic pieces 13 are attached onto the roller 12, and synchronously rotate with the roller 12. It should be noted that only two magnetic pieces 13 are shown for clearly explaining the aspects of the present invention, but not intended to limit the scope of the present invention. That is, the present invention may comprise any number of the magnetic pieces 12, and the magnetic pieces 12 may be arranged at different positions on the roller 12 to meet the actual application.

The circuit board 14 is fixed on the shell body 11 like the location over the shell body 11, but not limited. The battery unit 15 is provided on the circuit board 14 for supplying electric power, and may comprise a rechargeable battery such as lithium battery.

The sensor 16 is also on the circuit board 14, and near one side of the roller 12 like the location over the roller 12. In particular, the sensor 16 is not in contact with the roller 12 and the magnetic pieces 13, and receives the electric power from the battery unit 15 to operate. Furthermore, the sensor 16 detects variation of the magnetic flux caused by the magnetic pieces 13 to generate and transfer the electrical signal while the magnetic pieces 13 rotate. For instance, the sensor 16 comprises a Hall sensor, and the electrical signal is intended to indicate the speed of the air flow.

The blue tooth IC 17 is on the circuit board 14, electrically connected to the sensor 16 and the battery unit 15, and receives the electric power from the battery unit 15 to operate. Further, the blue tooth IC 17 receives the electrical signal from the sensor 16 and performs an inhalation counting process or a breathing capacity detection process to generate and transfer an inhalation counting result or a breathing capacity data.

More specifically, the above inhalation counting result or the breathing capacity data is wireless transferred to the external receiving processing device (not shown) by the blue tooth IC 17, and the external receiving processing device performs a data process to store and display the inhalation counting result or the breathing capacity data.

In addition, when the inhalator 20 is connected to the medicine bottle 30, if the switch 31 of the medicine bottle 30 is pressed by the inhalator 20 and opened, the medical agent or gas in the medicine bottle 30 immediately sprays into the inhalator 20, flows through the shell body 11, and finally is released from the inhalation end 11B to the outside. At this time, the roller 12 and the magnetic pieces 13 perform the low speed rotation, and the sensor 16 generates the electrical signal by detecting the variation of the magnetic flux caused by the magnetic pieces 13. The blue tooth IC 17 receives the electrical signal from the sensor 16 and counts to obtain the inhalation counting result, indicating the number of inhalation by the user. Or more specifically, the number of inhalation is calculated while the roller 12 operates at the low speed rotation mode for one time of inhaling the medical agent or gas. The above device 10 is considered to operate at an inhalation counting mode.

Alternatively, when the inhalator 20 is not connected to the medical bottle 30, if the user blows the inhalation end 11B, the roller 12 and the magnetic pieces 13 are dragged by the blow to perform the high speed rotation for a period of time, and the sensor 16 detects the variation the magnetic flux from the magnetic pieces 13 to generate the corresponding electrical signal. The blue tooth IC 17 receives the electrical signal to detect and calculate the breathing capacity, thereby obtaining the breathing capacity data. Substantially, the breathing capacity data represents the breathing capacity of the user corresponding to the blowing action. The above device 10 is considered to operate at a breathing capacity measuring mode.

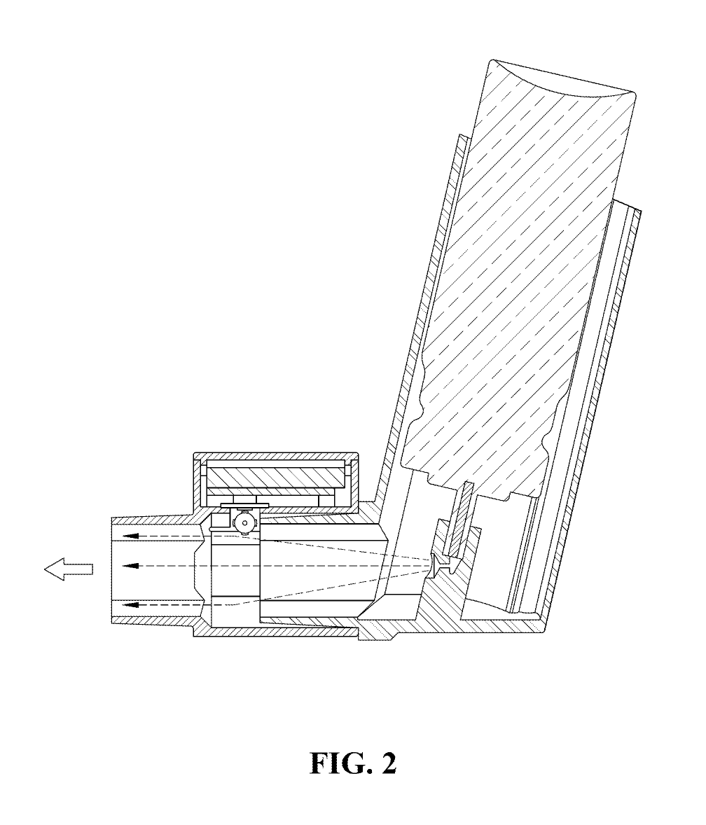

Further refer to FIG. 2. When the blue tooth IC 17 operates at the inhalation counting mode and the user inhales through the inhalation end 11B, the medical agent or gas in the medicine bottle 30 sprays into the mouth of the user through the inhalation end 11B of the inhalator 20, and the medical agent or gas is further transferred into the body of the user. At the same time, the blue tooth IC 17 counts based on the electrical signal from the sensor 16 to obtain the inhalation counting result indication the number of inhalation by the user.

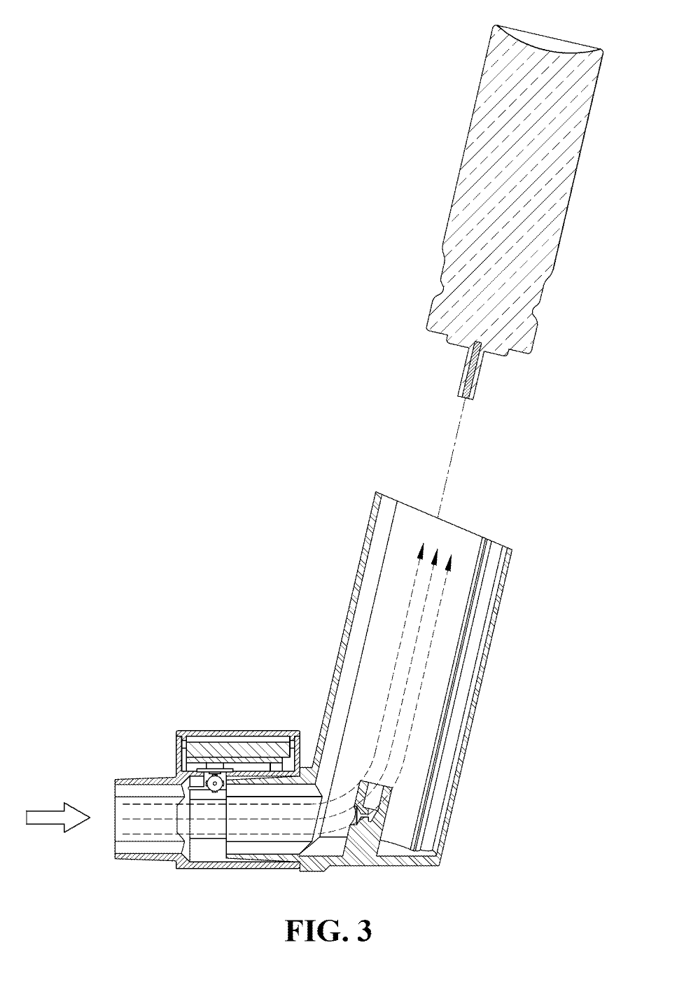

As shown in FIG. 3, when the medicine bottle 30 is not connected to the inhalator 20, the blue tooth IC 17 operates at the breathing capacity measuring mode, and the user is allowed to blow the inhalation end 11B. Because the inhalator 20 is not connected to the medicine bottle 30, the blowing air flow directly enters the inhalator 20 through the cavity 11A, and is discharged to the outside. It is apparently that the device 10 of the present invention is only allowed the air to pass and cause the roller 12 to rotate, and the blue tooth IC 17 receives and employs the electrical signal generated by the sensor 16 to detect and calculate the breathing capacity result, indicating the breathing capacity of the user.

In general, the breathing capacity measuring mode needs fast blowing, and is thus configured to operate at the high speed rotation. Relatively, the inhalation counting mode operates at the low speed rotation.

From the above mentioned, one of the key aspects provided by the present invention is to implement two operation modes, including the breathing capacity measuring mode at the high speed rotation and the inhalation counting mode at the low speed rotation, particularly, depending on whether the medicine bottle is connected or not. Specifically, the inhalation counting mode is performed when the medicine bottle is connected, and the breathing capacity measuring mode is performed when the medicine bottle is not connected.

In addition, the magnetic pieces can be in collocation with the sensor to construct a simple sensing unit for easily detect the number of inhalation or the breathing capacity by employing the air flow generated by inhalation or blowing. Owing to the simple overall structure, easy implementation and maintenance, and endurance, the present invention possesses excellent market competitiveness and industrial utility, and thus possibly replaces all traditional inhalators.

Although the present invention has been described with reference to the preferred embodiments thereof, it is apparent to those skilled in the art that a variety of modifications and changes may be made without departing from the scope of the present invention which is intended to be defined by the appended claims.

What is claimed is:

1. A device for measuring breathing capacity/counting inhalation detachably connected to an inhalator for accommodating a medicine bottle containing a medical agent or a gas at a preset pressure, the medicine bottle connecting or not connecting, and provided with a switch for restoring or releasing the medical agent or the gas, the device comprising:
a shell body forming a cavity, provided with a inhalation end for a user inhaling or exhaling, and a connection end for connecting an end of the inhalator, the other end of the inhalator connecting or not connecting the medicine bottle for the user inhaling or exhaling the shell body;
a roller provided on an inner wall of the cavity and dragged by an air flow through the cavity to perform a high speed rotation or a low speed rotation for a period of time;
a plurality of magnetic pieces attached onto and rotating synchronously with the roller;
a circuit board fixed to the shell body;
a battery unit provided on the shell body to supply electric power to the circuit board;
a sensor provided on the shell body and near the roller, but not in contact with the magnetic pieces for receiving the electric power from the battery unit to sense variation of a magnetic flux resulting from the magnetic pieces being rolling to generate and transfer an electrical signal; and
a blue tooth integrated circuit (IC) provided on the circuit board, electrically connected to the sensor and the battery unit, receiving the electric power from the battery unit, and performing an inhalation counting process or a breathing capacity detection process to generate and transfer an inhalation counting result or a breathing capacity data.

2. The device as claimed in claim 1, wherein the sensor comprises a Hall sensor.

3. The device as claimed in claim 1, wherein the inhalation counting result or the breathing capacity data is sent to an external receiving and processing device by means of a wireless transfer for performing a data process to store and display the inhalation counting result or the breathing capacity data.

4. The device as claimed in claim 1, wherein the switch is opened when the inhalator is connected with the medicine bottle and pressed, the medical agent or the gas in the medicine bottle is sprayed into the inhalator and further released to the inhalation end through the shell body, the roller in collocation with the magnetic pieces performs the low speed rotation, the sensor senses the variation of the magnetic flux from the magnetic pieces to generate the electrical signal, and the blue tooth IC receives the electrical signal to perform counting and obtain the inhalation counting result indicating a number of inhalation performed by the user.

5. The device as claimed in claim 1, wherein the user inhales through the inhalation end when the inhalator is not connected to the medicine bottle to cause the roller in collocation with the magnetic pieces to perform the high speed rotation, the blue tooth IC detects and calculates the breathing capacity based on the electrical signal to acquire the breathing capacity data indicating the breathing capacity of the user.
